(19) United States
(12) Patent Application Publication
(10) Pub. No.: US 2008/0166434 A1
(43) Pub. Date: Jul. 10, 2008

(54) Diet supplement for burning additional calories, providing sustained energy, supporting weight loss, and/or improving mental focus

(76) Inventor: Marvin A. Heuer, Mississauga (CA)

Correspondence Address: IOVATE HEALTH SCIENCE RESEARCH INC., 381 North Service Road West, Oakville, ON, L6M 0H4, omitted

(21) Appl. No.: 12/075,835

(22) Filed: Mar. 14, 2008

Related U.S. Application Data
(63) Continuation of application No. 11/447,476, filed on Jun. 5, 2006.
(60) Provisional application No. 60/688,420, filed on Jun. 7, 2005.

(51) Int. Cl. A61K 36/82; A61P 43/00

(52) U.S. Cl. 424/729

(57) ABSTRACT

A diet supplement comprising at least Green Tea Extract and Oolong Tea Extract may be in a time-release mechanism for sustained all-day energy, burning of calories, supporting weight loss, and improving mental focus. A method for providing sustained all-day energy, burning of calories, weight loss support, and improved mental focus by consumption of the diet supplement is also provided.

### RELATED APPLICATIONS

This application is a continuation of U.S. Ser. No. 11/447,476, filed Jun. 5, 2006, which claims the benefit of U.S. Provisional Patent Application No. 60/688,420, filed Jun. 7, 2005, entitled “Diet Supplement for Burning Additional Calories, Providing Sustained Energy, Supporting Weight Loss, and/or Improving Mental Focus”, the disclosure of which is hereby fully incorporated by reference.

### FIELD OF THE INVENTION

The present invention relates to a diet supplement for burning additional calories, providing sustained energy, supporting weight loss, and/or improving mental focus. Preferably, the diet supplement is provided in a time-release form for burning additional calories, providing sustained energy, supporting weight loss, and/or improving mental focus for an extended period of time throughout the day, e.g., an entire work day. In addition, the present invention relates to a method of promoting same by consuming the diet supplement.

### SUMMARY OF THE INVENTION

The present invention provides for a diet supplement that burns additional calories, provides sustained energy, supports weight loss, and/or improves mental focus. The diet supplement may include Caffeine Anhydrous. In addition or alternatively, the diet supplement may include one or more of Green Tea Dry Leaf Extract, White Tea Dry Leaf Extract and/or Oolong Tea Dry Leaf Extract. In one embodiment of the present invention, the diet supplement includes Green Tea Dry Leaf Extract and Caffeine Anhydrous in equal quantities. Advantageously, the diet supplement is provided in a time-release form, for burning additional calories, providing sustained energy, supporting weight loss, and/or improving mental focus for an extended period of time, e.g., up to 12 hours, so as to be an all-work day formula, after being consumed by an individual.

The present invention also provides, by the consumption of the supplemental composition, a method of burning additional calories, providing sustained energy, supporting weight loss, and/or improving mental focus.

### DETAILED DESCRIPTION OF THE INVENTION

The present invention, according to various embodiments thereof, is directed to a diet supplement that burns additional calories, provides sustained energy, supports weight loss, and/or improves mental focus.

Advantageously, the diet supplement is provided in a time-release form, for burning additional calories, providing sustained energy, supporting weight loss, and/or improving mental focus for an extended period of time after being consumed by an individual. For example, in an example embodiment, the diet supplement is provided in a time-release form that has a time release of approximately eight hours. Thus, for an embodiment that includes caffeine having a half-life of approximately 4 hours, each serving of the diet supplement may burn additional calories, provide sustained energy, support weight loss, and/or improve mental focus for up to 12 hours after being consumed by an individual. In this manner, the diet supplement may provide an all-work day formula in that it may burn additional calories, provide sustained energy, support weight loss, and/or improve mental focus throughout an entire “work-day” of a typical individual.

Green Tea Dry Leaf Extract

All teas of which the diet supplement of the present invention may be comprised, for example e.g., Green Tea, White Tea and Oolong Tea, are derived from the same plant namely Camellia sinensis. However, through the use of different processing methods, different proportions of active compounds result in each of the respective teas. White Tea undergoes very little processing, as does Green Tea, thereby leaving a relatively large amount of active compounds. Unlike green tea however, white tea is harvested before the leaves are fully opened. The processing of Oolong Tea is typically more involved than that of green tea. The active compounds of tea are a family of polyphenols, particularly the Catechins. The most active specific compound is the Catechin, epigallocatechin gallate (ECGC) which comprises from about 10 to about 50% of the total Catechins (Kao Y H, Hiipakka R A, Liao S. Modulation of endocrine systems and food intake by green tea epigallocatechin gallate. Endocrinology. 2000 March; 141(3):980-7). Furthermore, Green Tea also contains caffeine, although typically significantly less than Black Tea. Green tea and the active compounds isolated from Green Tea are the most widely studied teas to date.

The principal beneficial activity of Green Tea imparted by polyphenols is its antioxidant activity as evidenced by several studies. One clinical study has shown that ingestion of green tea extract results in a rapid increase in plasma antioxidant activity (Benzie I F, Szeto Y T, Strain J J, Tomlinson B. Consumption of green tea causes rapid increase in plasma antioxidant power in humans. Nutr Cancer. 1999; 34(1):83-7). Green tea has also been shown to be effective in aiding weight loss (Chantre P, Lairon D. Recent findings of green tea extract AR25 (Exolise) and its activity for the treatment of obesity. Phytomedicine. 2002 January; 9(1):3-8). This effect may be due to two activities. Firstly, Green Tea reduces fat digestion and secondly it increases energy expenditure (Berube-Parent S, Pelletier C, Dore J, Tremblay A. Effects of encapsulated green tea and Guarana extracts containing a mixture of epigallocatechin-3-gallate and caffeine on 24 h energy expenditure and fat oxidation in men. Br J Nutr. 2005 September; 94(3):432-6). The increase in energy expenditure may be derived from fat stores via the oxidation of fat, resulting in thermogenesis (Choo J J. Green tea reduces body fat accretion caused by high-fat diet in rats through beta-adrenoceptor activation of thermogenesis in brown adipose tissue. J Nutr Biochem. 2003 November; 14(11):671-6; Dulloo A G, Duret C, Rohrer D, Girardier L, Mensi N, Fathi M, Chantre P, Vandermander J. Efficacy of a green tea extract rich in catechin polyphenols and caffeine in increasing 24-h energy expenditure and fat oxidation in humans. Am J Clin Nutr. 1999 December; 70(6):1040-5). The thermogenic activity of Green Tea may additionally be greatly enhanced by its synergistic cooperation with caffeine (Dulloo A G, Seydoux J, Girardier L, Chantre P, Vandermander J. Green tea and thermogenesis: interactions between catechin-polyphenols, caffeine and sympathetic activity. Int J Obes Relat Metab Disord. 2000 February; 24(2):252-8). Moreover, an inverse relationship has also been demonstrated with respect to Green Tea consumption and total cholesterol levels (Kono S, Shinchi K, Ikeda N, Yanai F, Imanishi K. Green tea consumption and serum lipid profiles: a cross-sectional study in northern Kyushu, Japan. Prev Med. 1992 July; 21(4):526-31).

The mechanism of action for fat loss resulting from Green Tea consumption may be, at least partially, due to an increase in norepinephrine. Catechins are known to inhibit catechol-O-methyl-transferase (COMT), an enzyme that degrades norepinephrine (Borchardt R T, Huber J A. Catechol O-methyltransferase. 5. Structure-activity relationships for inhibition by flavonoids. J Med Chem. 1975 January; 18(1):120-2). In turn, norepinephrine inhibits degradation of intracellular cyclic AMP (cAMP), an important signaling molecule involved in many metabolic processes including thermogenesis. EGCG has been shown to be an inhibitor of glutamate dehydrogenase, which regulates insulin secretion (Li C, Allen A, Kwagh J, Doliba N M, Qin W, Najafi H, Collins H W, Matschinsky F M, Stanley C A, Smith T J. Green tea polyphenols modulate insulin secretion by inhibiting glutamate dehydrogenase. J Biol Chem. 2006 Apr. 14; 281(15):10214-21. Epub 2006 Feb. 13). The Anticancer activities associated with Green Tea may be related to its antioxidant activity and inhibition of vascular endothelial growth factor (VEGF) receptor signaling, which plays a role in tumor angiogenesis (Lamy S, Gingras D, Beliveau R. Green tea catechins inhibit vascular endothelial growth factor receptor phosphorylation. Cancer Res. 2002 Jan. 15; 62(2):381-5; Lee Y K, Bone N D, Strege A K, Shanafelt T D, Jelinek D F, Kay N E. VEGF receptor phosphorylation status and apoptosis is modulated by a green tea component, epigallocatechin-3-gallate (EGCG), in B-cell chronic lymphocytic leukemia. Blood. 2004 Aug. 1; 104(3):788-94. Epub 2004 Mar. 2).

The diet supplement may include Green Tea Dry Leaf Extract. In one embodiment of the present invention, which is set forth in greater detail in Example 1 below, the diet supplement may comprise Green Tea Dry Leaf Extract wherein a serving includes from about 1 mg to about 2000 mg of Green Tea Dry Leaf Extract. The preferred dosage of a serving of the diet supplement comprises about 600 mg of Green Tea Dry Leaf Extract.

Additionally, in a second embodiment of the present invention, which is set forth in greater detail in Example 2 below, the diet supplement may comprise Green Tea Dry Leaf Extract wherein a serving includes from about 1 mg to about 2000 mg of Green Tea Dry Leaf Extract. The preferred dosage of a serving of the diet supplement comprises about 598 mg of Green Tea Dry Leaf Extract.

In an embodiment of the present invention, the diet supplement comprises Green Tea Dry Leaf Extract, wherein the Green Tea Dry Leaf Extract comprises about 90% polyphenols. In one such embodiment of the present invention, the diet supplement includes Green Tea Dry Leaf Extract, wherein the Green Tea Dry Leaf Extract comprises about 75% Catechins. Furthermore, in one such embodiment of the present invention, the diet supplement comprises Green Tea Dry Leaf Extract, wherein the Green Tea Dry Leaf Extract comprises about 45% epigallocatechin gallate (“EGCG”).

In a second embodiment of the present invention, the diet supplement comprises Green Tea Dry Leaf Extract, wherein the Green Tea Dry Leaf Extract comprises about 98% polyphenols. In one such embodiment of the present invention, the diet supplement includes Green Tea Dry Leaf Extract, wherein the Green Tea Dry Leaf Extract comprises about 75% Catechins. Furthermore, in one such embodiment of the present invention, the diet supplement comprises Green Tea Dry Leaf Extract, wherein the Green Tea Dry Leaf Extract comprises about 45% epigallocatechin gallate (“EGCG”).

White Tea Dry Leaf Extract

White Tea is reported to have the same health benefits of green tea. However, White Tea has been reported to impart these benefits to an even greater extent (Santana-Rios G, Orner G A, Amantana A, Provost C, Wu S Y, Dashwood R H. Potent antimutagenic activity of white tea in comparison with green tea in the Salmonella assay. Mutat Res. 2001 Aug. 22; 495(1-2):61-74). White Tea has further been shown to possess anticarcinogenic properties in rats (Santana-Rios G, Orner G A, Xu M, Izquierdo-Pulido M, Dashwood R H. Inhibition by white tea of 2-amino-1-methyl-6-phenylimidazo[4,5-b]pyridine-induced colonic aberrant crypts in the F344 rat. Nutr Cancer. 2001; 41(1-2):98-103). A 6-month double blind, placebo controlled, randomized study on healthy post-menopausal females demonstrated that a supplement containing white tea improved skin condition, structure and firmness (Skovgaard G R, Jensen A S, Sigler M L. Effect of a novel dietary supplement on skin aging in post-menopausal women. Eur J Clin Nutr. 2006 May 3). White tea, as shown by a bacterial virus inactivation assay, has been reported to be more effective than Green Tea and to also possess anti-fungal properties (Dr. Schiffenbauer, Pace University, 104th General Meeting of the American Society for Microbiology on May 23, 2004, New Orleans, La.).

In addition or alternatively to the ingredients set forth above, the diet supplement may include White Tea Dry Leaf Extract. In an embodiment of the present invention, which is set forth in greater detail in Example 1 below, the diet supplement may comprise White Tea Dry Leaf Extract wherein a serving includes from about 0.1 mg to about 1000 mg of White Tea Dry Leaf Extract. The preferred dosage of a serving of the diet supplement comprises about 1.56 mg of White Tea Dry Leaf Extract.

In addition or alternatively to the ingredients set forth above, the diet supplement, in a second embodiment may include White Tea Dry Leaf Extract. In an embodiment of the present invention, which is set forth in greater detail in Example 2 below, the diet supplement may comprise White Tea Dry Leaf Extract wherein a serving includes from about 0.1 mg to about 1000 mg of White Tea Dry Leaf Extract. The preferred dosage of a serving of the diet supplement comprises about 1.00 mg of White Tea Dry Leaf Extract.

In both embodiments of the present invention, the diet supplement comprises White Tea Dry Leaf Extract, wherein the White Tea Dry Leaf Extract comprises about 50% polyphenols. In one such embodiment of the present invention, the diet supplement comprises White Tea Dry Leaf Extract, wherein the White Tea Dry Leaf Extract comprises about 35% Catechins. Furthermore, in one such embodiment of the present invention, the diet supplement comprises White Tea Dry Leaf Extract, wherein the White Tea Dry Leaf Extract comprises about 15% EGCG.

Oolong Tea Dry Leaf Extract Oolong Tea has also been specifically studied for beneficial activities. The chemopreventative activity has been demonstrated in rats (Matsumoto N, Kohri T, Okushio K, Hara Y. Inhibitory effects of tea catechins, black tea extract and oolong tea extract on hepatocarcinogenesis in rat. Jpn J Cancer Res. 1996 October; 87(10):1034-8) has been experimentally shown. Employing a comparative study in rats, it was found that Oolong Tea was superior in controlling weight as compared to Green Tea, while Green Tea was more effective at lowering total cholesterol (Kuo K L, Weng M S, Chiang C T, Tsai Y J, Lin-Shiau S Y, Lin J K. Comparative studies on the hypolipidemic and growth suppressive effects of oolong, black, pu-erh, and green tea leaves in rats. J Agric Food Chem. 2005 Jan. 26; 53(2):480-9). As a method of weight control, clinical trials in humans have shown that Oolong Tea increases metabolic rate and energy expenditure (Rumpler W, Seale J, Clevidence B, Judd J, Wiley E, Yamamoto S, Komatsu T, Sawaki T, Ishikura Y, Hosoda K. Oolong tea increases metabolic rate and fat oxidation in men. J Nutr. 2001 November; 131(11):2848-52; Komatsu T, Nakamori M, Komatsu K, Hosoda K, Okamura M, Toyama K, Ishikura Y, Sakai T, Kunii D, Yamamoto S. Oolong tea increases energy metabolism in Japanese females. J Med Invest. 2003 August; 50(3-4):170-5). Clinical trials have further demonstrated the potential therapeutic benefit of Oolong Tea as a treatment for diabetes (Hosoda K, Wang M F, Liao M L, Chuang C K, Iha M, Clevidence B, Yamamoto S. Antihyperglycemic effect of oolong tea in type 2 diabetes. Diabetes Care. 2003 June; 26(6):1714-8) and coronary artery disease (Shimada K, Kawarabayashi T, Tanaka A, Fukuda D, Nakamura Y, Yoshiyama M, Takeuchi K, Sawaki T, Hosoda K, Yoshikawa J. Oolong tea increases plasma adiponectin levels and low-density lipoprotein particle size in patients with coronary artery disease. Diabetes Res Clin Pract. 2004 September; 65(3):227-34).

In addition or alternatively to the ingredients set forth above, the diet supplement may include Oolong Tea Dry Leaf Extract. In an embodiment of the present invention, which is set forth in greater detail in Example 1 below, the diet supplement may comprise Oolong Tea Dry Leaf Extract wherein a serving includes from about 0.1 mg to about 1000 mg of Oolong Tea Dry Leaf Extract. The preferred dosage of a serving of the diet supplement comprises about 1.56 mg of Oolong Tea Dry Leaf Extract.

In addition or alternatively to the ingredients set forth above, the diet supplement, a second embodiment may include Oolong Tea Dry Leaf Extract. In an embodiment of the present invention, which is set forth in greater detail in Example 2 below, the diet supplement may comprise Oolong Tea Dry Leaf Extract wherein a serving includes from about 0.1 mg to about 1000 mg of Oolong Tea Dry Leaf Extract. The preferred dosage of a serving of the diet supplement comprises about 1.00 mg of Oolong Tea Dry Leaf Extract.

In both embodiments of the present invention that are set forth above, the diet supplement comprises Oolong Tea Dry Leaf Extract, wherein the oolong tea dry leaf extract comprises about 50% polyphenols. In one such embodiment of the present invention, the diet supplement comprises Oolong Tea Dry Leaf Extract, wherein the Oolong Tea Dry Leaf Extract comprises about 25% Catechins. Furthermore, in one such embodiment of the present invention, the diet supplement comprises Oolong Tea Dry Leaf Extract, wherein the Oolong Tea Dry Leaf Extract comprises about 15% EGCG.

Anhydrous Caffeine

Anhydrous Caffeine is a naturally occurring xanthine alkaloid found in some plants, where it acts as a natural pesticide. In humans, however, it may have numerous beneficial effects, the most common of which uses caffeine as a supplement to the central nervous system. In this capacity, it is used as a stimulant and performance enhancer. Biochemically, caffeine which is structurally similar to adenosine receptors, binds to, but does not activate, adenosine receptors which are normally activated by adenosine to induce sleep (Shi D, Nikodijevic O, Jacobson K A, Daly J W. Chronic caffeine alters the density of adenosine, adrenergic, cholinergic, GABA, and serotonin receptors and calcium channels in mouse brain. Cell Mol Neurobiol. 1993 June; 13(3):247-61). This antagonism of adenosine receptors leads to increased levels of neurotransmitters.

A meta-analysis compiled from forty double-blind studies support the use of Caffeine to increase physical endurance (Doherty M, Smith P M. Effects of caffeine ingestion on exercise testing: a meta-analysis. Int J Sport Nutr Exerc Metab. 2004 December; 14(6):626-46; Graham T E, Hibbert E, Sathasivam P. Metabolic and exercise endurance effects of coffee and caffeine ingestion. J Appl Physiol. 1998 September; 85(3):883-9; Kovacs E M, Stegen J H C H, Brouns F. Effect of caffeinated drinks on substrate metabolism, caffeine excretion, and performance. J Appl Physiol. 1998 August; 85(2):709-15). Furthermore, Caffeine is also widely used to control weight, which may occur through multiple mechanisms. Significant weight loss has been observed in obese women related to caffeine supplementation (Yoshida T, Sakane N, Umekawa T, Kondo M. Relationship between basal metabolic rate, thermogenic response to caffeine, and body weight loss following combined low calorie and exercise treatment in obese women. Int J Obes Relat Metab Disord. 1994 May; 18(5):345-50) which may be, at least in part, due to increased lipolysis as fat is metabolized (Jung R T, Shetty P S, James W P, Barrand M A, Callingham B A. Caffeine: its effect on catecholamines and metabolism in lean and obese humans. Clin Sci (Lond). 1981 May; 60(5):527-35). Caffeine has also been shown to increase metabolic rate in both lean and obese individuals (Roberts A T, de Jonge-Levitan L, Parker C C, Greenway F. The effect of an herbal supplement containing black tea and caffeine on metabolic parameters in humans. Altern Med Rev. 2005 December; 10(4):321-5; Astrup A, Toubro S, Cannon S, Hein P, Breum L, Madsen J. Caffeine: a double-blind, placebo-controlled study of its thermogenic, metabolic, and cardiovascular effects in healthy volunteers. Am J Clin Nutr. 1990 May; 51(5):759-67; Dulloo A G, Geissler C A, Horton T, Collins A, Miller D S. Normal caffeine consumption: influence on thermogenesis and daily energy expenditure in lean and postobese human volunteers. Am J Clin Nutr. 1989 January; 49(1):44-50) wherein this adds to its weight-lowering effects. Furthermore, caffeine additionally improves cognitive performance following physical activity (Hogervorst E, Riedel W J, Kovacs E, Brouns F, Jolles J. Caffeine improves cognitive performance after strenuous physical exercise. Int J Sports Med. 1999 August; 20(6):354-61).

In addition or alternatively to the ingredients set forth above, the diet supplement may include Caffeine Anhydrous. In an embodiment of the present invention, which is set forth in greater detail in Examples 1 and 2 below, the diet supplement may comprise Caffeine Anhydrous wherein a serving includes from about 1 mg to about 2000 mg of Caffeine Anhydrous. The preferred dosage of a serving of the diet supplement comprises about 400 mg of Caffeine Anhydrous. Furthermore, in an embodiment of the present invention, the diet supplement includes green tea dry leaf extract and caffeine anhydrous in equal quantities.

The present invention, according to various embodiments thereof, provides a method of burning additional calories, providing sustained energy, supporting weight loss, and/or improving mental focus by the consumption of the diet supplement. Advantageously, consumption of the diet supplement is combined with a program of diet and exercise.

According to various embodiments of the present invention, the diet supplement may be consumed in any form. For instance, the dosage form of the diet supplement may be provided as, e.g., a powder beverage mix, a liquid beverage, a ready-to-eat bar or drink product, a capsule, a tablet, a caplet, or as a dietary gel. The most preferred dosage form is a caplet.

Preferably, the diet supplement is consumed by an individual in accordance with the following: As a diet supplement, 2 caplets may be taken with an 8 oz. glass of water within one hour after waking up in the morning. More than two caplets should not be consumed by an individual in a 24-hour period. To assess individual tolerance, an individual may consume, on Day 1 to Day 3, 1 caplet daily. Thereafter, an individual may consume two caplets daily.

Furthermore, the dosage form of the diet supplement may be provided in accordance with customary processing techniques for herbal and/or dietary supplements in any of the forms mentioned above.

The diet supplement of the present invention may be provided in a time release mechanism. U.S. Pat. No. 5,445,826, entitled “Delivery System Containing a Gel-Forming Fiber and a Drug” discloses a prolonged-release dosage formulation preferably in a tablet form. The patent purports to describe a composition that includes a gel-forming fiber, preferably hydrocolloid-coated, a biologically-absorbable drug, or other active therapeutic agent which is also preferably hydrocolloid-coated, and a mineral salt such as mineral carbonate or bicarbonate which releases a physiologically-acceptable gas such as carbon dioxide upon ingestion. The composition may optionally also contain phosphoric acid and a dextrose or similar sugar. The aforementioned fiber-containing coating, when in the form of a tablet or other unit dosage form together with the drug or agent, provides a controllable prolonged action drug-delivery system.

U.S. Pat. No. 5,292,518, entitled “Prolonged-Release Drug Table Formulations” discloses a prolonged-release unit dosage formulation or pharmaceutical composition. Preferably, the unit dosage is in the form of a table wherein the composition consists of a gel-forming dietary fiber, a biologically-absorbable drug or other active therapeutic agent, and a disintegrant such a mineral salt e.g. mineral carbonate or bicarbonate, which releases a physiological acceptable gas such as carbon dioxide upon ingestion, and advantageously dextrose or similarly soluble sugar. Furthermore, a physiologically-acceptable acid may optionally be included in the composition to further facilitate the disintegration of the tablet. The dietary fiber-containing composition, when compressed into a table together with the drug and specific disintegrant, provides a prolonged-action drug-delivery system.

U.S. Pat. No. 5,096,714 entitled “Prolonged-release Drug Tablet Formulations” purports to describe a composition to provide a prolonged-action drug-delivery system. The invention comprises a composition consisting of a gel-forming dietary fiber, a biologically-absorbable drug or other active therapeutic agent, disintegrants such as physiological-acceptable edible acids and mineral salts; which upon ingestion release a physiological acceptable gas such as carbon dioxide, as well as dextrose or a similarly soluble sugar. The unit dosage according to the present invention is a tablet.

A study designed to assess the effectiveness of the pharmacokinetics of extended release Caffeine tablets was performed. The study was a single-site, open label, phase 1 study involving 30 healthy subjects with no known allergies or hypersensitivity to Caffeine. Subjects first visited the study site for an initial screening and to consent to the study and on a second occasion to the clinic for Caffeine dosing. According to the dosage schedule, subject was asked to refrain from Caffeine intake for 48 hours prior to the second visit.

Subjects were dosed with 600 mg Caffeine in extended-release capsules and blood sample were taken at 0, 0.5, 1, 2, 3, 6, 8, 10, 11, and 12 hours via an 18-gauge arm-inserted catheter. Urine was collected at 0, 6, and 12 hours.

The half-life of the extended-release Caffeine capsule for the pooled subjects was 7.09 hours. Five subjects had kinetic that did not allow for a calculation of the half-life and were excluded from the pooled subjects. As a comparison, the accepted half-life for Caffeine in a non-extended release format is 3.5 to 5 hours. Utilizing the extend-release format, the release period is approximately 70% longer that the non-extended release format. Furthermore, the maximum concentration of the Caffeine in the serum was 5.76 mg/l with a Tₘₐₓ median and mode of 3 hours.

In the present invention, two examples of which are set forth in greater detail in Examples 1 and 2 below, a diet supplement is provided for burning additional calories, providing sustained energy, supporting weight loss, and/or improving mental focus. In this manner, consumption by an individual of the supplemental composition provides for a method for burning additional calories, providing sustained energy, supporting weight loss, and/or improving mental focus. According to the example embodiment set forth below, the diet supplement is provided and consumed in the form of a time-release tablet.

The diet supplement set forth in the example embodiment below may contain one or more of the following excipients: guar gum, dicalcium phosphate, calcium carbonate, microcrystalline cellulose, stearic acid, vegetable stearin, citrus pectin, magnesium stearate, silica and film coating (hypromellose, hydroxypropyl cellulose, and polyethylene glycol).

Although the following examples illustrate the practice of the present invention in two of its embodiments, the examples should not be construed as limiting the scope of the invention. Other embodiments will be apparent to one skilled in the art from consideration of the specification of the following examples.

### EXAMPLES

### Example 1

A diet supplement formula for promoting the burning of additional calories, providing sustained energy, supporting weight loss, and/or improves mental focus is provided comprising Green Tea Dry Leaf Extract (0.60000 g) standardized to 45% EGCG, 75% Catechins, 90% Polyphenols, Anhydrous Caffeine (0.40000 g), White Tea Dry Leaf Extract (0.00156 g) standardized to 15% EGCG, 35% Catechins, 50% Polyphenols, and Oolong Tea Dry Leaf Extract (0.00156 g) standardized to 15% EGCG, 25% Catechins, 50% Polyphenols. The present embodiment, taken as a daytime supplement, may provide sustained energy, improve mental focus as well as support weight loss by adding in the burning of additional calories.

Directions: As a dietary supplement, 2 caplets may be taken with an 8 oz. glass of water within one hour following waking in the morning.

### Example 2

A diet supplement formula for promoting the burning of additional calories, providing sustained energy, supporting weight loss, and/or improves mental focus is provided comprising Green Tea Dry Leaf Extract (0.59800 g) standardized to 45% EGCG, 75% Catechins, 90% Polyphenols, Anhydrous Caffeine (0.40000 g), White Tea Dry Leaf Extract (0.00100 g) standardized to 15% EGCG, 35% Catechins, 50% Polyphenols, and Oolong Tea Dry Leaf Extract (0.00100 g) standardized to 15% EGCG, 25% Catechins, 50% Polyphenols. The present embodiment, taken as a daytime supplement, may provide sustained energy, improve mental focus as well as support weight loss by adding in the burning of additional calories.

Directions: As a dietary supplement, 2 caplets may be taken with an 8 oz. glass of water within one hour following waking in the morning.

What is claimed:

1. A diet supplement comprising Green Tea Extract and Oolong Tea Extract.

2. The diet supplement of claim 1, further comprising White Tea Extract.

3. The diet supplement of claim 2, wherein the Green Tea Extract is Green Tea Dry Leaf Extract, the White Tea Extract is White Tea Dry Leaf Extract, and the Oolong Tea Extract is Oolong Tea Dry Leaf Extract.

4. The diet supplement of claim 3, further comprising Anhydrous Caffeine.

5. The diet supplement of claim 4, further comprising a time-release mechanism.

6. The diet supplement of claim 5, wherein the time-release mechanism provides for up to and including 12 hours active compound release.

7. The diet supplement of claim 4, wherein the Green Tea Dry Leaf Extract is present in amounts from about 0.01 g to about 2 g, the Anhydrous Caffeine is present in amounts from about 0.001 g to about 1 g, the White Tea Dry Leaf Extract is present in amounts from about 0.0001 g to about 0.01 g and the Oolong Tea Dry Leaf Extract is present in amounts from about 0.0001 to about 0.01 g per serving.

8. The diet supplement of claim 4, wherein the Green Tea Dry Leaf Extract, Anhydrous Caffeine, White Tea Dry Leaf Extract and Oolong Tea Dry Leaf Extract are present in amounts effective for at least one of burning calories, providing sustained energy, supporting weight loss and improving mental focus in a human or animal.

9. A method for at least one of burning calories, providing sustained energy, supporting weight loss and improving mental focus in a human or animal, comprising the step of administering to the human or animal a diet supplement that comprises Green Tea Extract and Oolong Tea Extract.

10. The method of claim 9, wherein the diet supplement further comprises White Tea Extract.

11. The method of claim 10, wherein the Green Tea Extract is Green Tea Dry Leaf Extract, the White Tea Extract is White Tea Dry Leaf Extract, and the Oolong Tea Extract is Oolong Tea Dry Leaf Extract.

12. The method of claim 11, wherein the diet supplement further comprises Anhydrous Caffeine.

13. The method of claim 12, wherein the diet supplement is provided in a time release mechanism.

14. The method of claim 13, wherein the time-release mechanism provides for up to and including 12 hours active compound release.

15. The method of claim 12, wherein the diet supplement is administered to the human or animal within at least one hour of waking in the morning.
